(19) United States
(12) Patent Application Publication
(10) Pub. No.: US 2009/0036768 A1
(43) Pub. Date: Feb. 5, 2009

(54) MEDICAL DEVICES

(75) Inventors: Ashley Seehusen, Newton, MA (US); Christopher Nardone, N. Chelmsford, MA (US); Mark Girard, Medway, MA (US); Mark L. Adams, Sandy, UT (US); Todd Beaupre, Reading, MA (US); Raymond J. Lareau, Westford, MA (US)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 11/941,314

(22) Filed: Nov. 16, 2007

Related U.S. Application Data
(60) Provisional application No. 60/866,242, filed on Nov. 17, 2006.

(51) Int. Cl. A61B 6/12; A61M 29/02; A61M 25/098; B05D 5/00

(52) U.S. Cl. 600/424; 606/200; 604/529; 427/2.11

(57) ABSTRACT

Medical devices with radiopaque markers, and related systems and methods are disclosed.

### CROSS-REFERENCE TO RELATED APPLICATIONS

This application claims priority under 35 U.S.C. §119 to U.S. Ser. No. 60/866,242, filed Nov. 17, 2006, the contents of which are hereby incorporated by reference.

### FIELD

This disclosure relates to medical devices with radiopaque markers, and related systems and methods.

### BACKGROUND

Medical devices with radiopaque markers can be placed within body lumens, and radiopaque markers can help to ensure that the medical devices are positioned accurately. Examples of medical devices include catheters, embolic coils, and guidewires.

### SUMMARY

In one aspect, the invention generally features a medical device that includes an elongated body having a groove and a radiopaque material in the groove.

In another aspect, the invention generally features a medical device that includes an elongated body having a groove and a material in the groove, wherein a difference between a maximum outer diameter of the device along a length of the groove and a maximum outer diameter of the device adjacent to the groove is at most about 10% of a maximum thickness of the elongated body.

In a further aspect, the invention generally features a medical device that includes an elongated body and a coating that includes a radiopaque material. The coating is supported by the elongated body, and the coating has a groove.

In an additional aspect, the invention features a method of making a medical device. The method includes providing an elongated body of the medical device, determining a desired distance between radiopaque markers to be associated with the elongated body and associating the radiopaque markers with the elongated body to form the medical device. The radiopaque markers are spaced a distance that is within six mils of the desired distance.

In yet another aspect, the invention generally features a method of making a medical device. The method includes disposing a radiopaque material on an elongated body of the medical device, and forming a groove in the radiopaque material.

Embodiments can include one or more of the following features.

The elongated body can be tube-shaped.

The medical device can be, for example, an embolic coil, a guide wire or a catheter.

A difference between a maximum outer diameter of the medical device along a length of a groove and a maximum outer diameter of the medical device adjacent to the groove can be at most about 10% of a maximum thickness of the elongated body.

The groove can have a width of at least 0.040 mm.

The groove can have a depth of at least 0.1 mm.

The radiopaque material can form a portion of an outer surface of the medical device.

The elongated body can include a polymer and/or a metal-containing material (e.g., a shape memory alloy, such as, for example, nitinol).

The elongated body can have a plurality of grooves. The plurality of grooves can form a hatched pattern.

The medical device can further include a coating supported by the elongated body and the radiopaque material.

The radiopaque material can be selected from, for example, bismuth-containing materials, metals and alloys.

The elongated body can have a groove in its inner surface and a groove in its outer surface.

The groove can extend into the elongated body.

The method can include forming grooves in the elongated body and disposing a respective radiopaque marker in each groove.

The method can further include forming a groove in the elongated body.

Embodiments of the invention can include any of the following advantages.

In some embodiments, one or more grooves machined in a surface of a medical device can be filled with a radiopaque material so that a surface profile of the medical device is substantially smooth. Such medical devices can provide the benefits of having desired radiopacity (e.g., the ability to accurately position within a body lumen) along with the benefits of a smooth surface profile (e.g., easier to dispose and accurately position within small body lumens than medical devices with bumps and/or other surface profile features).

In certain embodiments, a position of radiopaque markers on the medical devices may be substantially unchanged when a coating material is deposited on a surface of the medical devices. For example, in certain embodiments, radiopaque markers are positioned securely within grooves formed in the surface of the medical devices, and shrinkage or other movement of one or more coating layers does not change a position of the markers.

In some embodiments, radiopaque markers can be accurately positioned with respect to one another on a surface of a medical device. For example, positions of grooves in which radiopaque markers are positioned can be machined in surfaces of medical devices with high accuracy, so that distances between markers are known and do not change significantly over time. Accurate positioning of markers can assist in ensuring accurate placement of the devices within body lumens.

In certain embodiments, widths and/or depths of grooves machined in surfaces of medical devices can be selected to control visibility of radiopaque markers in imaging (e.g., x-ray imaging) of the medical devices. For example, a width and/or depth of a groove can be selected to control an amount of radiopaque material deposited in the groove, and thereby to control the visibility of the filled groove.

In some embodiments, multiple radiopaque markers can be provided in a portion of a medical device to increase the visibility of that portion of the device in imaging (e.g., x-ray imaging). For example, multiple grooves filled with radiopaque material can be provided at selected positions along a length of a medical device so that those positions can be accurately identified on x-ray images of the device in a body lumen. Further, multiple grooves can form patterns on portions of a surface of the device, making visual identification of the patterned portions easier in images (e.g., x-ray images) of the device.

In certain embodiments, grooves machined in a surface of a medical device can impart flexibility to the device, without significantly reducing torsional strength of the device. For example, by imparting flexibility to the medical device, the device can be formed from materials such as certain metals which would otherwise be too stiff for use in the device. These materials can have other advantageous properties such as corrosion resistance, for example.

In some embodiments, grooves can be machined into a surface of a medical device after a radiopaque coating has been applied to the surface. By depositing the radiopaque coating before machining the device, grooves are not occluded with excess coating material.

Other features and advantages of the invention will be apparent from the description, drawings, and claims.

### BRIEF DESCRIPTION OF THE DRAWINGS

FIG. 1A is a cross-sectional view of an embodiment of a catheter with grooves filled with radiopaque material.

FIG. 1B is a cross-sectional view of the catheter shown in FIG. 1A taken along line 1B-1B.

FIG. 1C is a cross-sectional view of the catheter shown in FIG. 1A taken along line 1C-1C.

FIG. 2 is a cross-sectional view of an embodiment of a catheter with grooves having angled side walls.

FIG. 3 is a cross-sectional view of an embodiment of a catheter with rounded grooves.

FIG. 4 is a plan view of an embodiment of a catheter having grooves that extend only partially along a circumference of the catheter.

FIG. 5 is a plan view of an embodiment of a catheter having both longitudinal and circumferential grooves.

FIG. 6 is a side view of the catheter of FIG. 5.

FIG. 7 is a plan view of an embodiment of a catheter having patterns formed by groups of circumferential grooves.

FIG. 8 is a plan view of an embodiment of a catheter having a cross-hatched pattern of grooves.

FIG. 9 is a plan view of an embodiment of a catheter having helical grooves that form a cross-hatched pattern.

FIG. 10 is a plan view of an embodiment of a catheter having grooves that do not extend completely around the circumference of the catheter.

FIG. 11 is a cross-sectional view of an embodiment of a catheter with grooves in an inner surface of the catheter body.

FIG. 12 is a cross-sectional view of an embodiment of a catheter with grooves in inner and outer surfaces of the catheter body.

FIG. 13 is a cross-sectional view of an embodiment of a catheter having a coating.

FIG. 14 is a cross-sectional view of an embodiment of a catheter having grooves formed in a coating material.

Like reference symbols in the various drawings indicate like elements.

### DETAILED DESCRIPTION

This disclosure relates to medical devices, such as, for example, catheters, embolic coils, guidewires, that include a radiopaque material. The radiopaque material can assist in accurately positioning the devices within body lumens.

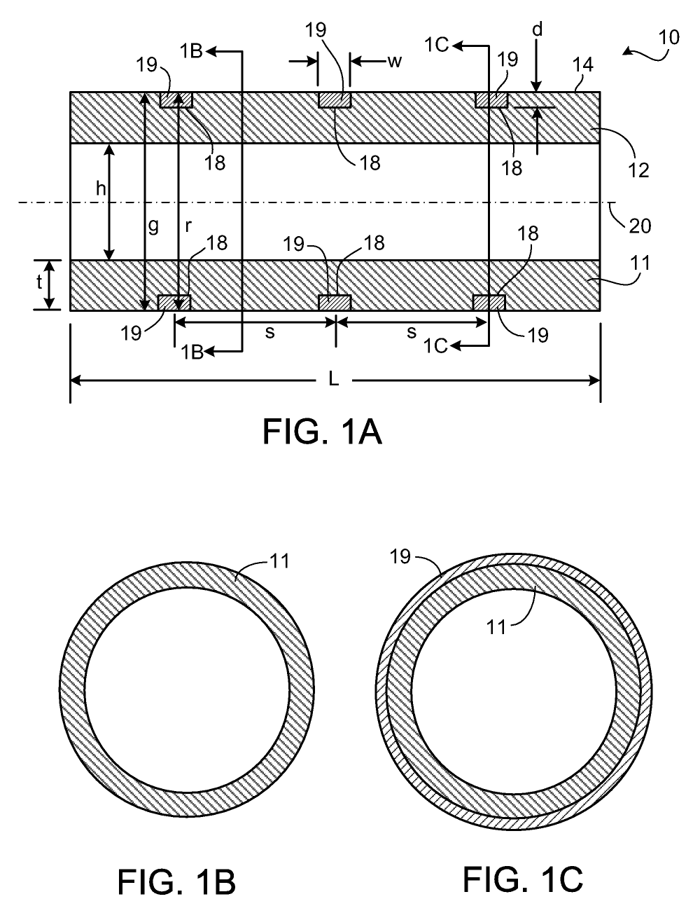

FIGS. 1A-1C are cross-sectional views of a catheter 10. Catheter 10 has an elongated, tubular body 11 having a length L, thickness t, outer diameter g, and inner diameter h. Body 11 has an inner surface 12, an outer surface 14 and grooves 18. Grooves 18 extend around the circumference of body 11 and have a depth d measured along a radial direction transverse to longitudinal axis 20 of body 11 and a width w measured along a direction parallel to axis 20. Grooves 18 are spaced from one another by an amount s measured in a direction parallel to axis 20. Grooves 18 are filled with a radiopaque material 19 so that the outer diameter of catheter 10 along a length of the grooves 18 is r.

In some embodiments, body 11 can be formed of a metal-containing material, such a metal or alloy. Examples of metal containing materials include stainless steel, aluminum, magnesium, and shape memory alloys. Examples of shape memory materials include nitinol, silver-cadmium alloys, gold-cadmium alloys, gold-copper-zinc alloys, copper-aluminum-nickel alloys, copper-gold-zinc alloys, copper-zinc alloys, copper-zinc-aluminum alloys, copper-zinc-tin alloys, copper-zinc-xenon alloys, iron beryllium (Fe₃Be), iron platinum (Fe₃Pt), indium-thallium alloys, iron-manganese alloys, nickel-titanium-vanadium alloys, iron-nickel-titanium-cobalt alloys and copper-tin alloys. In some embodiments, body 11 can be formed from a polymer material such as a polyamide, such as a nylon material (e.g., PEBAX), a polyurethane material, a polycarbonate material, or another type of polymer material. In certain embodiments, mixtures of materials can be used to form body 11. For example, mixtures of polymer materials (e.g., polyurethanes and polyamides) can be used to form body 11.

The length L of catheter 10 can generally be selected as desired according to the function of catheter 10. In some embodiments, L can be 1 mm or more (e.g., 5 mm or more, 10 mm or more, 20 mm or more, 30 mm or more, 40 mm or more). In certain embodiments, L can be 300 cm or less (e.g., 200 cm or less, 100 cm or less, 50 cm or less, 10 cm or less). As an example, in some embodiments in which catheter 10 is an ocular drainage shunt, L can be 1 mm. As another example, in certain embodiments in which catheter 10 is employed in endoscopic use, L can be 300 cm.

In general, the widths w and depth d of grooves 18 can be selected to provide particular mechanical properties to catheter 10. For example, grooves 18 having relatively large widths w and/or depths d can impart a relatively large amount of flexibility to catheter 10 along axis 20. Grooves 18 having relatively small widths w and/or depths d can impart a relatively small amount of flexibility to catheter 10 along axis 20. In addition, by controlling a length of grooves 18 along a circumference of catheter 10, the torsional strength of catheter 10 can be controlled. In general, the shorter the length of grooves 18 along the circumference of catheter 10, the greater the torsional strength of catheter 10.

In some embodiments, the depth d of grooves 18 can be 0.1 mm or more. For example, d can be 0.2 mm or more (e.g., 0.3 mm or more, 0.4 mm or more, 0.5 mm or more). In certain embodiments, d can be 5 mm or less (e.g., 4 mm or less, 3 mm or less, 2 mm or less, 1 mm or less). In general, d is less than t; that is, grooves 18 do not extend in a radial direction fully from outer surface 14 to inner surface 12 of catheter 10.

In some embodiments, the depth d of grooves 18 can be at least 5% or more of the maximum thickness t of tubular body 11. For example, d can be at least 10% or more (e.g., at least 20% or more, at least 30% or more, at least 40% or more, at least 50% or more) of the thickness t of tubular body 11. In certain embodiments, d can be 95% or less (e.g., 90% or less, 85% or less, 80% or less, 75% or less, 70% or less) of the thickness t of tubular body 11.

In some embodiments, the width w of grooves 18 can be 3 mm or more. For example w can be 3.5 mm or more (e.g., 4 mm or more, 4.5 mm or more, 5 mm or more). In certain embodiments, w can be 2 mm or less. For example, w can be 1.5 mm or less (e.g., 1 mm or less, 0.5 mm or less, 0.05 mm or less, 0.045 mm or less).

While in the embodiment of catheter 10 shown in FIGS. 1A-1C, the widths w and depths d of grooves 18 are the same, more generally, the widths w and/or depths d of grooves 18 can vary among grooves. For example, widths and depths of grooves 18 can be varied to control a volume of radiopaque material that fills each groove 18. As an example, a lower volume groove can contain less radiopaque material, which can reduce the visibility of that portion of catheter 10 in x-ray images. As another example, a higher volume groove can contain more radiopaque material, which can increase the visibility of that portion of catheter 10 in x-ray images. Thus, by controlling the widths w and depths d of grooves 18, the visibility of the grooves can be controlled. This provides a method for shading catheter 10, whereby certain radiopaque material-filled grooves appear darker than others in x-ray images.

In some embodiments, the spacings s between grooves 18 can be chosen to be regular, (e.g., so that grooves 18 form a regular marker array extending along axis 20). For example, the spacing s can be 3 mm or more (e.g., 4 mm or more, 5 mm or more, 6 mm or more, 10 mm or more). In certain embodiments, the spacing s can be 30 mm or less (e.g., 25 mm or less, 20 mm or less, 15 mm or less, 10 mm or less).

In certain embodiments, the spacings s can be chosen to vary the mechanical properties of catheter 10. In particular, the spacings s can be selected to increase the ability of catheter 10 to bend at positions along axis 20. At the same time, grooves 18 can be configured so that they do not significantly reduce the torsional strength of catheter 10 about axis 20. Therefore, grooves 18 can be provided to adjust the mechanical properties of catheter 10, which can allow for the use of certain materials such as some metal-containing materials (e.g., nitinol, aluminum, stainless steel) that might otherwise be too stiff to use in catheters designed for implantation in body lumens where significant bending is required to maneuver the catheter into place.

The spacings s between grooves 18 shown in FIGS. 1A-1C are the same. However, in some embodiments, s can vary between adjacent grooves along axis 20. For example, in certain embodiments, multiple grooves can be positioned in close proximity in a particular region of catheter 10, such as a distal or proximal end of catheter 10. The multiple grooves can be positioned with relatively small spacings between adjacent grooves. Additional grooves can be located at other positions along axis 20, for example, with relatively large spacings along axis 20 between adjacent grooves, so that the closely spaced groups of grooves can function as specific position markers, and the longer spaced grooves can function as a ruler or scale in x-ray images. As another example, in certain embodiments, multiple grooves can be positioned in close proximity in a particular region of catheter 10 in order to increase the flexibility of that region of catheter 10. The spacings between these adjacent grooves along axis 20 can be relatively small. In other regions of catheter 10, spacings between adjacent grooves along axis 20 can be relatively large, so that the flexibility of catheter 10 is not significantly changed.

In some embodiments, the reproducibility of the spacings s between grooves can be important to ensure that catheter 10 is positioned accurately. This can, for example, enhance the ability to accurately place catheter 10 within a body lumen. In some embodiments, the difference between a desired (nominal) groove spacing determined prior to fabrication and an actual groove spacing in catheter is less than 6 mils (e.g., less than 5 mils, less than 4 mils, less than 3 mils, less than 2 mils, less than 1 mil, less than 0.5 mil).

Examples of radiopaque materials include bismuth-containing materials (e.g., bismuth trioxide, bismuth bicarbonate, bismuth oxychloride, and other bismuth-containing materials), metals (e.g., tungsten, tantalum, platinum, palladium, lead, gold, silver, titanium, and other metals), alloys (e.g., stainless steel, tungsten-containing alloys, tantalum-containing alloys, platinum-containing alloys, palladium-containing alloys, lead-containing alloys, gold-containing alloys, silver-containing alloys, titanium-containing alloys, and other alloys), metal oxides (e.g., titanium dioxide, zirconium dioxide, aluminum oxide, and other oxides), barium-containing materials (e.g., barium sulfate and other barium-containing materials), radiopaque contrast agents (e.g., Omnipaque™, Renocal®, iodiamide meglumine, diatrizoate meglumine, ipodate calcium, ipodate sodium, iodamide sodium, iothalamate sodium, iopamidol, metrizamide, and other contrast agents), and other materials. In some embodiments, the same radiopaque material can be used to fill each of the grooves 18. In certain embodiments, some grooves can be filled with radiopaque materials that are different from the radiopaque materials used to fill other grooves. For example, different grooves can be filled with different concentrations and/or types of radiopaque materials to vary the visibility of the filled grooves with respect to one another. Certain grooves can be filled with a large concentration of highly radiopaque material to make those filled grooves highly visible in x-ray images. Other grooves can be filled with a lower concentration of less radiopaque material so that the filled grooves are visible in x-ray images, but not as highly visible as grooves filled with highly radiopaque material. This permits a further type of radiopaque shading of portions of catheter 10.

Body 11 is typically formed by first forming a tube, and then forming grooves 18 to provide body 11 having grooves 18. In some embodiments, grooves 18 are formed using diamond saw machining of the outer surface of the tube. An example of an apparatus that can be used is disclosed, for example, in U.S. Pat. No. 6,014,919, which is hereby incorporated by reference. The widths w, depths d, and spacings s of the grooves can be very accurately controlled during such a machining process. The accuracy to which these dimensions are controlled is typically higher than would be possible using certain machining methods such as grit blasting and ordinary saw cutting. However, various methods may be used to form grooves 18, depending on a desired degree of accuracy.

Various techniques can be used to fill grooves 18 with radiopaque material. In some embodiments, outer surface 14 can be masked with a masking agent so that only grooves 18 remain exposed, and then one or more radiopaque materials can be deposited in grooves 18 using chemical or physical vapor deposition techniques. Removing the masking agent following deposition yields a catheter 10 having a smooth outer surface 14, with grooved portions filled with radiopaque material so that the outer diameter r of catheter 10 along grooves 18 is substantially equal to the outer diameter g of catheter 10 adjacent to grooves 18.

In certain embodiments, grooves 18 can be filled by wrapping circular sections of wire that include radiopaque material into grooves 18, such that the sections of wire are positioned within grooves 18 and do not extend outward from grooves 18 in a radial direction further than outer surface 14. In some embodiments, the sections of wire can be single sections that extend around the entire circumference of catheter 10. In certain embodiments, multiple sections of wire can be used to fill grooves 18. For example, two semi-circular sections of wire can be used to fill grooves 18. The semi-circular sections of wire can be shaped so that they snap securely into grooves 18. Following installation, the semi-circular sections of wire can be bonded to one another, if desired. The diameter or thicknesses of the wire sections can be selected to ensure that a cross-sectional width of the wire is similar to the width w of grooves 18, and a thickness of the wire is similar to the depth d of grooves 18, so that grooves 18 are substantially filled by the wire sections. The outer diameter r of catheter 10 along grooves 18 can be substantially equal to the outer diameter g of catheter 10 adjacent to grooves 18.

In some embodiments, grooves 18 can be filled with radiopaque material using other techniques. For example, radiopaque material can be processed into a moldable gel, and the gel can be injected into grooves 18 to fill the grooves. Curing and/or further processing the injected gel produces a catheter 10 having filled grooves 18, where the outer diameter r of catheter 10 along grooves 18 is substantially equal to the outer diameter g of catheter 10 adjacent to grooves 18.

Many of the lumens in which catheter 10 can be placed have small diameters, and it can be desirable for the outer surface of catheter 10 (defined by the exposed surfaces of body 11 and radiopaque material 19) to be as smooth as possible so that catheter 10 can be inserted and positioned within a body lumen as easily as possible. A smooth outer surface can be maintained if grooves 18 are filled so that differences between the outer diameter r of catheter 10 along the length(s) of the groove(s) and the outer diameter g of catheter 10 adjacent to the groove(s) is/are relatively small. In some embodiments, the filling process for grooves 18 yields a catheter 10 where a difference between a maximum outer diameter of catheter 10 along a length of a groove and a maximum outer diameter of catheter 10 adjacent to the groove is at most about 10% of the maximum thickness t of tubular body 11. For example, the difference between the maximum outer diameter of catheter 10 along the length of the groove, r, and the maximum outer diameter of catheter 10 adjacent to the groove, g, can be at most about 5% (e.g., at most about 1%, at most about 0.1%, at most about 0.01%, at most about 0.001%) of the maximum thickness t of tubular body 11.

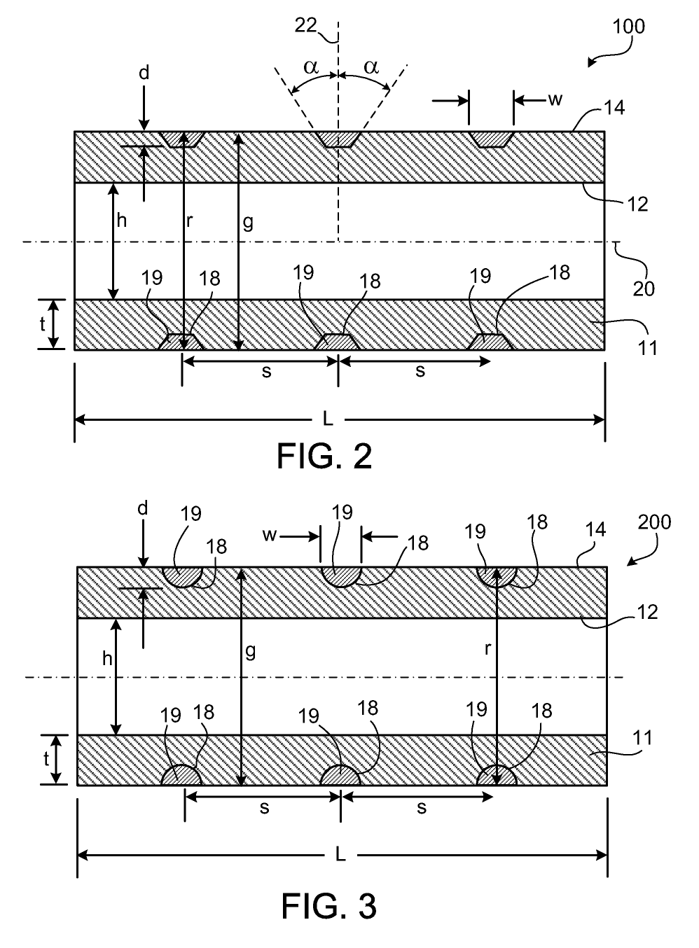

In general, in embodiments in which grooves 18 are formed using a diamond saw, the cross-sectional shape of grooves 18 is at least partially determined by the shape of the diamond saw. For example, in some embodiments, grooves 18 can have a cross sectional shape where the walls of grooves 18 are angled with respect to radial lines of catheter 10. As an example, FIG. 2 is a cross-sectional view of a catheter 100 with grooves 18 having walls that are angled with respect to a radial line 22 of catheter 100. Grooves 18 have a maximum width w measured along a direction parallel to axis 20, and a depth d measured along a radial direction perpendicular to axis 20. Widths w and depths d can be the same as disclosed above, for example. An angle α of the walls of grooves 18 with respect to radial line 22 can be determined by the machining process, for example. The magnitude of angle α can be, for example, 0° or more (e.g., 10° or more, 20° or more) and/or 90° or less (e.g., 60° or less, 30° or less).

In some embodiments, surfaces of grooves 18 can be rounded. As an example, FIG. 3 is a cross-sectional view of a catheter 200 having rounded grooves 18. Grooves 18 have a width w measured along a direction parallel to axis 20, and a maximum depth d measured along a radial direction perpendicular to axis 20. Rounded grooves 18 can be advantageous, for example, where sections of cylindrical wire containing radiopaque material are used to form radiopaque markers by filling grooves 18.

FIGS. 2 and 3 show two different embodiments of non-rectangular groove cross-sectional shapes. However, a wide variety of different shapes are possible, depending upon the specific fabrication processes used to form catheter 10. Other examples of groove cross-sectional shapes include triangular grooves, and tilted rectangular grooves (e.g., grooves with rectangular cross-sectional shapes where the entire rectangular shape is oriented at an angle with respect to both longitudinal axis 20 and radial lines perpendicular to longitudinal axis 20).

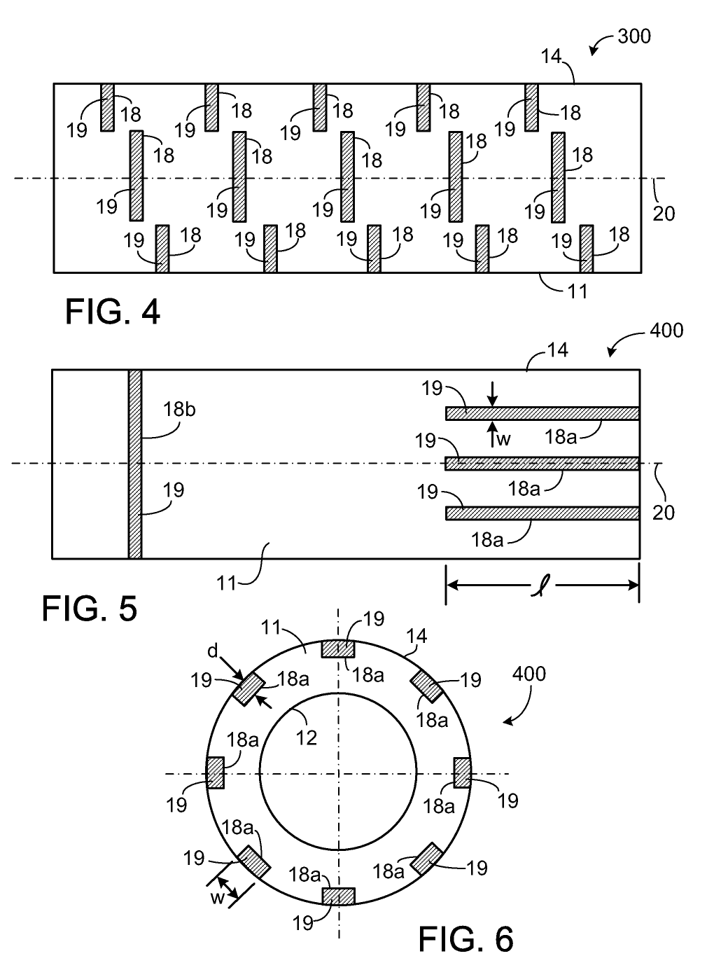

In some embodiments, grooves 18 extend around an entire circumference of catheter 10, as shown in FIGS. 1A-1C, 2, and 3. However, in certain embodiments, some or all of grooves 18 can extend along only a part of a circumference of catheter 10. For example, FIG. 4 shows a plan view of a catheter 300 having grooves 18 that extend along only a portion of the circumference of catheter 300. In general, the length of grooves 18 along a circumference of catheter 300 can be selected during the machining process. For example, grooves 18 can extend along 95% or less of the circumference of catheter 300 (e.g., 90% or less, 85% or less, 80% or less, 75% or less, 70% or less, 65% or less). In some embodiments, grooves 18 can extend along 10% or more of the circumference of catheter 300 (e.g., 20% or more, 25% or more, 30% or more).

In some embodiments, grooves 18 can be formed so that they extend parallel to axis 20 of catheter 10. For example, FIGS. 5 and 6 show plan and side views, respectively, of a catheter 400 having longitudinal grooves 18a extending parallel to axis 20. Longitudinal grooves 18a have widths w measured along a circumference of catheter 400. Longitudinal grooves 18a also have depths d measured along a radial direction, perpendicular to axis 20. Widths w and depths d can have the same values as disclosed previously, for example. Grooves 18a have lengths 1 measured along a direction parallel to axis 20 of catheter 400. For example, in some embodiments, 1 can be 0.5 mm or more (e.g., 1 mm or more, 5 mm or more, 10 mm or more, 15 mm or more, 20 mm or more). In certain embodiments, length 1 can be 20 cm or less (e.g., 15 cm or less, 10 cm or less, 5 cm or less).

Catheter 400 can also include one or more circumferential grooves 18b, as described previously. In some embodiments, groups of grooves 18a and 18b can alternate along axis 20 of catheter 400. The dimensions of each of grooves 18a and 18b can be independently controlled to adjust mechanical properties of catheter 400. For example, the dimensions of grooves 18a can be adjusted to control a reduction in torsional strength of catheter 400, and the dimensions of grooves 18b can be adjusted to control an increase in flexibility of catheter 400 along axis 20.

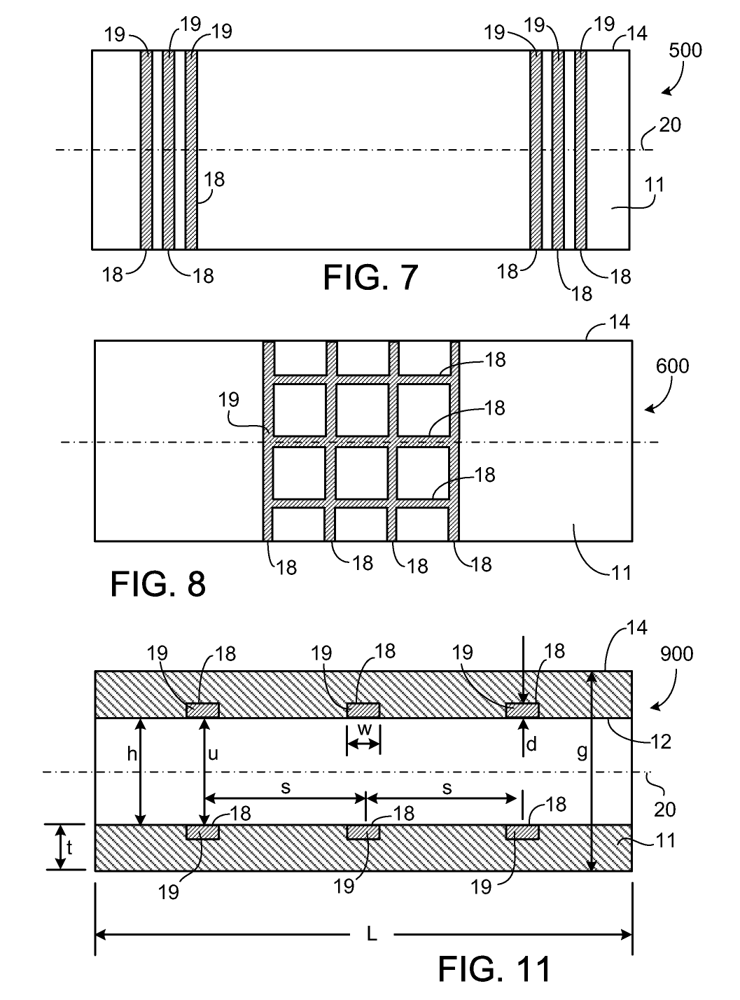

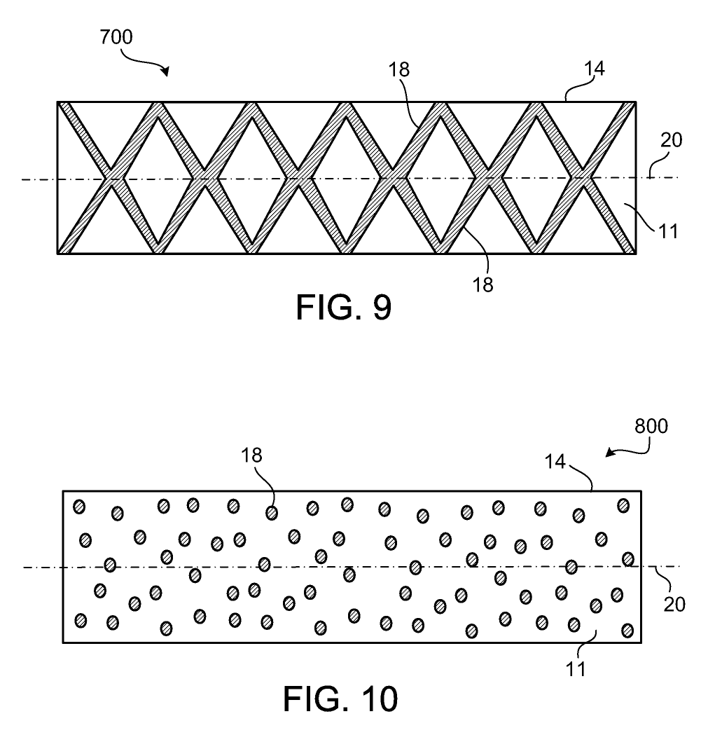

In some embodiments, grooves 18 can be positioned on catheter 10 to form patterns. For example, FIGS. 7-10 show plan views of catheters 500, 600, 700, and 800, respectively, where a plurality of grooves 18 form patterns on outer surface 14 of the catheters. In FIG. 7, groups of grooves 18 form multiple ring patterns in selected portions of catheter 500 along axis 20. The groups of grooves can be used to increase the visibility of the selected portions of catheter 500 in x-ray images. In FIG. 8, groups of circumferential and longitudinal grooves are positioned to form a cross-hatched pattern on portions of outer surface 14 of catheter 600. The pattern can increase the visibility of the cross-hatched portions of catheter 600 in x-ray images, and assist in visual identification of specific marked portions. In FIG. 9, multiple helical grooves 18 form a cross-hatched pattern on outer surface 14 of catheter 700. As in FIGS. 7 and 8, the helical grooves in the embodiment of FIG. 9 can be used to increase the visibility of selected portions of catheter 700 in x-ray images. In FIG. 10, grooves 18 do not extend all the way around the circumference of catheter 800, but instead form islands in outer surface 14. The islands can be arranged in regular patterns (e.g., a rectangular array of islands) or in irregular patterns. In general, a wide variety of groove patterns can be produced on catheter surfaces, and the grooves can be filled with any of a variety of radiopaque materials. Multiple radiopaque patterns can be formed on a single catheter, and the multiple patterns can be used to distinguish different regions of the catheter in x-ray images. Further, in some embodiments, groove patterns are produced over substantially all of a surface (e.g., outer surface 14 and/or inner surface 12) of a catheter. In other embodiments, groove patterns cover only a portion of one or more surfaces of a catheter, to assist in distinguishing particular portions of the catheter in x-ray images.

While certain embodiments have been described, other are possible.

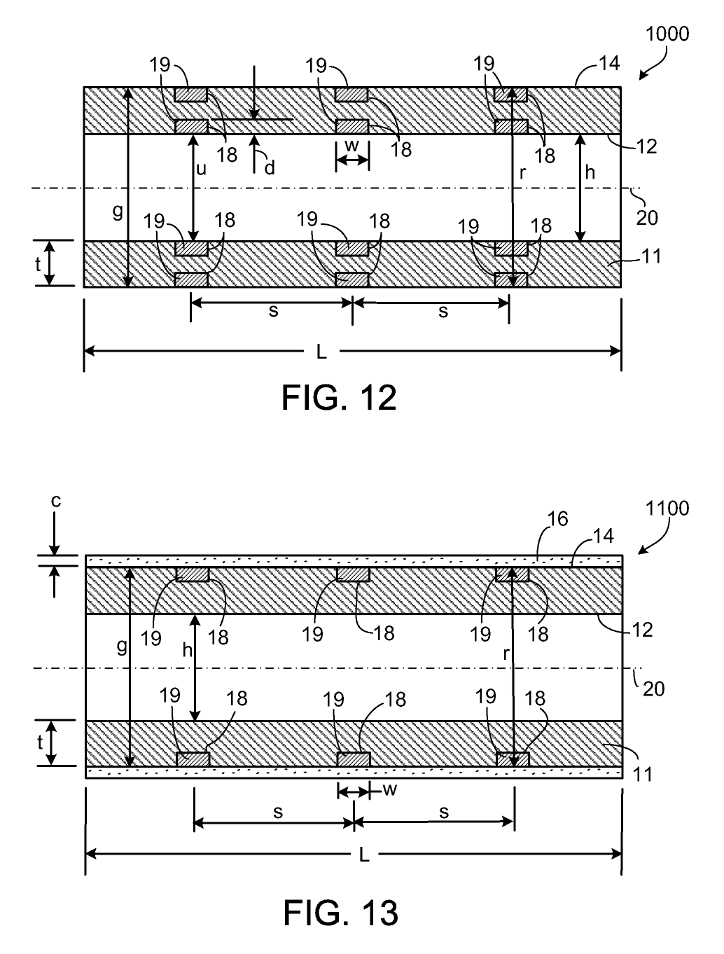

As an example, in some embodiments, grooves 18 can be formed along an inner circumference of body 11, such as shown, for example, in FIG. 11. In certain embodiments, grooves 18 can be formed along both the inner and outer circumferences of body 11, such as shown, for example, in FIG. 12. Grooves 18 along the inner circumference of body 11 can have the same widths w and spacings s measured along a direction parallel to axis 20, and depths d measured along a radial direction perpendicular to axis 20, as disclosed above. Grooves 18 along the inner circumference of body 11 can also have cross-sectional shapes similar to the cross-sectional shapes disclosed above. Grooves 18 along the inner circumference of body 11 can be filled with one or more of the radiopaque materials 19 as disclosed above.

In embodiments with one or more grooves formed along an inner circumference of body 11, a smooth inner surface of the catheter can be achieved by ensuring that differences between the inner diameter u of the catheter along the length(s) of the groove(s) and the inner diameter h adjacent to the groove(s) is/are relatively small. In certain embodiments, a difference between a maximum inner diameter u of the catheter along a length of a groove and a maximum inner diameter h of the catheter adjacent to the groove is at most about 10% (e.g., at most about 5%, at most about 1%, at most about 0.1%, at most about 0.01%, at most about 0.001%) of the maximum thickness t of the tubular body of the catheter.

In embodiments where grooves 18 are present along the inner and outer circumferences of body 11, grooves 18 can have the same features or different features. In general, any of the features of grooves disclosed above can be shared by grooves along the inner and outer circumferences of body 11. Alternatively, or in addition, grooves 18 along the inner and outer circumferences of body 11 can differ in regard to any of the groove features disclosed above. For example, in some embodiments, the cross-sectional shapes of grooves 18 along the inner and outer circumferences of body 11 can be similar, but the spacings between grooves 18 along the inner circumference of body 11 can be different than the spacings between grooves 18 along the outer circumference of body 11. As another example, different radiopaque materials 19 can be used to fill grooves 18 along the inner and outer circumferences of body 11. As a further example, grooves 18 along the inner circumference of body 11 can have the same spacings as grooves 18 along the outer circumference of body 11, but the grooves 18 along the inner circumferences of body 11 can be offset from grooves 18 along the outer circumference of body 11 in a direction parallel to axis 20. In general, many different combinations of similar and differing features of grooves are possible with respect to grooves 18 along the inner and outer circumferences of body 11.

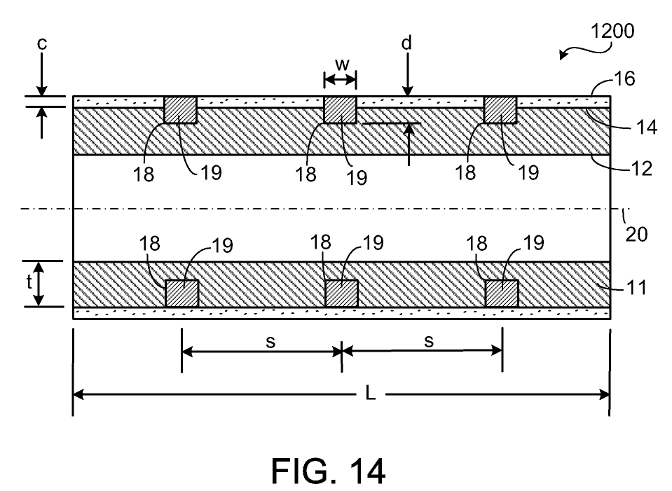

As another example, in certain embodiments a coating can be formed on the body of a catheter. For example, as shown in FIG. 13, a coating 16 can be present. In some embodiments, coating material 16 can be desirable to prevent the body material and/or the radiopaque material from contacting an interior of a body lumen. Examples of coating materials include polymer materials such as polyurethanes, nylons (e.g., PEBAX), polycarbonates, polyamides, and other polymer materials, for example. Coating materials can be hydrophilic materials (e.g., Bioslide, Hydropass, Hydrolene, Glidex, Tecogel, Aquaglide, and other hydrophilic coatings). In certain embodiments, multiple coating materials can be used. For example, multiple layers of the coating materials can be deposited in succession over outer surface 14 to impart desired physical characteristics to catheter 10. In some embodiments, one or more coating materials can be heat-shrunk or cured to ensure that coating 16 adheres tightly to outer surface 14. During these processes, coating 16 can shrink both longitudinally (e.g., in a direction parallel to axis 20) and radially (e.g., in a direction perpendicular to axis 20). In certain embodiments, coating material 16 can be deposited on outer surface 14 using various techniques such as spray-coating, dipping, painting, rollering, sponging, or other processes. In certain embodiments, coating 16 can be deposited prior to machining grooves 18 in catheter 10. For example, FIG. 14 shows a cross-sectional view of a catheter 1200 that includes a tubular catheter body formed from a length L and thickness t of catheter material, as described in connection with catheter 10. After the catheter body is formed, coating 16 is deposited atop outer surface 14 of catheter 1200. Grooves 18 are then machined into the body and coating 16 using the methods disclosed above. Subsequently, radiopaque material 19 is filled into grooves 18 and optionally also into the openings in coating 16. Grooves 18 have widths w measured along a direction parallel to axis 20, and depths d measured along a radial direction perpendicular to axis 20. In general, widths w and depths d can have the values disclosed previously. Coating 16 has a thickness c measured along a radial direction perpendicular to axis 20. In some embodiments, grooves 18 have depths d that are smaller than c. In other embodiments, grooves 18 have depths d that are larger than c, and extend radially inward into the tubular body of catheter 1200, as shown in FIG. 14.

As a further example, while embodiments of a catheter have been described, in general, other medical devices can be similarly designed. Examples of other medical devices include embolic coils and guidewires. As an example, radiopaque material-filled grooves 18 can be present in embolic coils. In such embodiments, visualization of the radiopaque material can, for example, assist in accurately positioning the embolic coils when they are inserted by a surgeon in a body lumen to occlude the lumen. As another example, radiopaque material-filled grooves can be present in guidewires. The filled grooves can assist in observing the location of the guidewire within a body lumen. Alternatively or additionally, the grooves can be used to control mechanical properties of an embolic coil or a guidewire. For example, the grooves can be designed to enhance the flexibility of an embolic coil or a guidewire along their longitudinal axes. This can be desirable, for example, when a medical device is to be inserted into small body lumen.

Other embodiments are in the claims.

1. A medical device, comprising:
an elongated body of the medical device, the elongated body having a groove; and
a radiopaque material in the groove.

2. The medical device of claim 1, wherein the elongated body is tube-shaped.

3. The medical device of claim 1, wherein the medical device is an embolic coil.

4. The medical device of claim 1, wherein the medical device is a catheter.

5. The medical device of claim 1, wherein the radiopaque material forms a portion of an outer surface of the medical device.

6. The medical device of claim 5, wherein the difference between a maximum outer diameter of the device along a length of the groove and a maximum outer diameter of the device adjacent to the groove is at most 10% of a maximum thickness of the elongated body.

7. The medical device of claim 1, wherein the groove has a width of at least 0.040 mm.

8. The medical device of claim 1, wherein the groove has a depth of at least 0.1 mm.

9. The medical device of claim 1, wherein the elongated body comprises a polymer.

10. The medical device of claim 1, wherein the elongated body comprises a metal-containing material.

11. The medical device of claim 1, wherein the elongated body comprises a shape memory alloy.

12. The medical device of claim 1, wherein the elongated body comprises nitinol.

13. The medical device of claim 1, wherein the elongated body has a plurality of grooves.

14. The medical device of claim 13, wherein the plurality of grooves form a hatched pattern.

15. The medical device of claim 1, further comprising a coating supported by the elongated body and the radiopaque material.

16. The medical device of claim 1, wherein the radiopaque material is selected from the group consisting of bismuth-containing materials, metals and alloys.

17. The medical device of claim 1, wherein the elongated body has a groove in its inner surface and a groove in its outer surface.

18. A medical device, comprising:
an elongated body of the medical device, the elongated body having a groove; and
a material in the groove,
wherein the difference between a maximum outer diameter of the device along a length of the groove and a maximum outer diameter of the device adjacent to the groove is at most 10% of a maximum thickness of the elongated body.

19. A medical device, comprising:
an elongated body of the medical device; and
a coating comprising a radiopaque material,
wherein the coating is supported by the elongated body, and the coating has a groove.

20. The medical device of claim 19, wherein the groove extends into the elongated body.

21. The medical device of claim 19, wherein the elongated body comprises a polymer.

22. The medical device of claim 19, wherein the elongated body comprises a metal-containing material.

23. The medical device of claim 22, wherein the radiopaque material is selected from the group consisting of bismuth-containing materials, metals and alloys.

24. The medical device of claim 19, wherein the radiopaque material is selected from the group consisting of bismuth-containing materials, metals and alloys.

25. A method of making a medical device, the method comprising:
providing an elongated body of the medical device;
determining a desired distance between radiopaque markers to be associated with the elongated body; and
associating the radiopaque markers with the elongated body to form the medical device, the radiopaque markers being spaced a distance that is within six mils of the desired distance.

26. The method of claim 25, further comprising:
forming grooves in the elongated body; and
disposing a respective radiopaque marker in each groove.

27. A method of making a medical device, the method comprising:
disposing a radiopaque material on an elongated body of the medical device; and
forming a groove in the radiopaque material.

28. The method of claim 27, further comprising forming a groove in the elongated body.

29. The method of claim 27, wherein the radiopaque material is selected from the group consisting of bismuth-containing materials, metals and alloys.

30. The method claim 27, wherein the elongated body comprises a metal-containing material.
